(19) United States
(12) Patent Application Publication
(10) Pub. No.: US 2021/0244913 A1
(43) Pub. Date: Aug. 12, 2021

(54) FLUID DELIVERY CATHETER

(71) Applicant: Allurion Technologies, Inc., Natick, MA (US)

(72) Inventor: David W. NELSON, Wayland, MA (US)

(73) Assignee: Allurion Technologies, Inc., Natick, MA (US)

(21) Appl. No.: 17/170,626

(22) Filed: Feb. 8, 2021

Related U.S. Application Data
(60) Provisional application No. 62/971,600, filed on Feb. 7, 2020.

(51) Int. Cl. A61M 25/00; A61M 25/10; A61F 5/00; B29C 61/02

(52) U.S. Cl. CPC A61M 25/0023; A61M 25/1018; A61F 5/003; B29L 2031/7543; A61M 2207/00; A61M 2025/0098; B29C 61/025

(57) ABSTRACT

Fluid delivery systems comprising catheters used to deliver a fluid into a medical device to which the catheter is attached. Methods and devices for eliminating the effects angular strain on the catheters that lead to kinking and pinching of the catheter.

### RELATED APPLICATION

This application is a non-provisional of U.S. Provisional application No. 62/971,600 filed on Feb. 7, 2020, the entirety of which is incorporated by reference herein.

### BACKGROUND

The present disclosure relates to fluid delivery systems that use a catheter or tubing to deliver fluids to a medical device.

FIG. 1 is an illustration of a conventional balloon device 100, specifically a gastric balloon for weight reduction, positioned in a patient's stomach. Balloon devices generally comprises two states: a pre-deployment or uninflated configuration and a deployed, inflated, or active configuration. Generally, a fluid delivered through a tube 110, inflates the device 100, where the tube 110 can also be referred to as a catheter or conduit. The tube may pass through an opening 115 in wall 102 of the balloon device 100. Alternatively, as shown, the tube 110 can be coupled to a fluid path 112, which fluidly connects the exterior and the interior of the balloon device. The end of catheter 110 that delivers the fluid to the interior reservoir of the balloon is the delivery end 110A while the opposite end is the fill end 110B, into which fluid is introduced.

In many balloon devices 100, a wall 102 of the balloon is fabricated from a thin film material such as, for example, polyurethane. In some variations, tube 110 comprises a balloon, or delivery, end 110A that extends through fluid path 112 into a central enclosed space or reservoir 104 of device 100. Conduit 110 is removed from the device once inflation is completed. When catheter 110 is removed, fluid path 112 must be sealed to prevent the inflation fluid from leaking out through fluid path 112 from reservoir 104. Again, in some variations, a fill valve, not illustrated, seals the device 100. In some variations the fill valve or the fluid path 112 acts to constrain tube 110 to pass through wall 102 at a fixed angle relative to the local normal to the wall. In some variations the angle is 90 degrees (that is, tube 110 is normal to wall 102) while in other variations tube 110 may pass through wall 102 at a shallower angle, even approaching 0 degrees.

Prior to the balloon being filled, thin film wall 102 is flexible. When tube 110 is constrained to pass through wall 102 at a fixed angle, any movement of tube 110 affects, bends, or distorts wall 102 such that the angle at which tube 110 passes through wall 102 is constant. FIG. 2A illustrates the nominal configuration of a tube 110 passing through an opening 115 in a section of wall 102 of thin film material that defines balloon device 100. Tube 110 passes through a constraining element 116, which may be, for example, a fluid path or fill valve. In this illustration tube 110 has an axis 110Z, which is constrained by element 116 to be parallel to the surface normal 102N of wall 102.

As further illustrated in FIG. 2B, when there is no significant pressure within device 100 wall 102 shape is distorted when tube 110 is pulled to one side so that the parallel relationship between tube axis 110Z and wall normal 102N is maintained. On the other hand, as balloon device 100 is filled with a fluid the internal pressure in device 100 increases and wall 102 experiences increasing tension. In turn, the increasing tension stiffens wall 102 making it resistant to distortion. In particular, after balloon 100 is inflated close to capacity, wall 102 is placed in tension and becomes relatively stiff. FIG. 3 illustrates that tensioned wall 102 has limited ability to tilt locally to maintain the surface normal 102N parallel to the catheter axis 110Z. Instead, catheter 110′ must bend to when the fill end 110B′ is deflected to the side. There is a limit to how far catheter 110′ may be pulled to the side before the decreasing radius of the curvature at the bend point reaches a critical radius, R_{C}. Any additional pull on the catheter causes the inner side of the curved catheter to fold or kink, shown in tube 110′. This folding reduces the fluid flow through the catheter and may weaken tubular wall 117 of the catheter. For highly flexible tube structures such as the catheter, R_{C} is small, and kinking occurs very close to the constraining element, illustrated as collar 116, between the tube 110 and the much less flexible balloon wall 102, that is, kinking in a tube occurs where there is a sharp discontinuity in the effective stiffness of the tube. This discontinuity can be eliminated by an angular strain-relief component 10, as shown in FIG. 3.

In some instances, the fill valve and/or the fluid path 112 may be designed to include angular strain relief. Angular Strain Relief is a means of reinforcing a generally flexible, linear component—a wire or tube—that is attached to a stiff and somewhat fixed attachment point to prevent the linear component from being damaged or kinked by a lateral force, that is, being pulled by a force directed perpendicular to the linear component's axis.

In the case of a flexible tube like a catheter, the kinking that occurs because of the lateral force is well understood. As explained in Mechanical Properties of Catheters (Acta Radiologica: Diagnosis, 4:sup260, 11-22) incorporated by reference herein, a straight catheter held fixed at one end and subjected to a force perpendicular to its axis takes on a curvature with a radius

$$R = \frac{{EI}_{o}}{M} \qquad ( 1 )$$

where

E is the modulus of elasticity of the catheter material,

Iₒ is the moment of inertia of the catheter with respect to its normal axis, and

M is the bending moment (that is, force applied to bend) applied to the catheter.

For a fixed M, the radius can be increased by changing the material to one with a higher modulus of elasticity (that is, a fixed applied force will bend a stiffer material less) or changing the geometry of the catheter to increase the moment of inertia. For a tube,

$${I}_{o} = \frac{π ( {D}^{4} - {d}^{4} )}{64} , \qquad ( 2 )$$

where D is the outer diameter of the catheter and d is its inner diameter. Clearly, the radius R depends strongly on the wall thickness (D−d)/2. For a catheter with a fixed inner diameter the wall thickness increases linearly with outer diameter D.

Appendix A further explains the critical radius. The critical radius, R_{C}, is the smallest radius into which the catheter can be bent before it kinks (reducing or stopping fluid flow through the catheter). From the appendix,

R_{C}=K(D²/(D−d)). (3)

where the scaling factor K is nearly constant for all catheter materials of interest. As a general rule it is desirable to have a small critical radius, which allows one to bend a catheter sharply without kinking. In any particular use, the catheter inner and outer diameter are selected to achieve the required R_{C}, with the critical radius generally decreasing with decreasing outer diameter (the inner diameter is typically fixed to achieve the desired fluid flow at a fixed pressure).

### Angular Strain Relief Variations

As described above, a tube will kink as the bending radius decreases to become equal to the critical radius. While it is possible to stiffen the catheter by increasing the outer diameter of the entire catheter to make it harder to reach the critical radius, it is usually more desirable to maintain high flexibility over most of the length of the catheter to facilitate placement through a tortuous path that must be navigated between outside the body and the device's ultimate operational location. Thus, the purpose of an angular strain relief to prevent the catheter's bending radius from reaching the critical radius in the immediate vicinity of the device, where the catheter is angularly constrained by the connection to the device wall, while maintaining the flexibility of the majority of the length of the catheter.

An angular strain relief acts to reduce the inherent discontinuity between the stiff constraining element and the flexible catheter. The strain relief, in one variation, is designed to provide a transition zone along the catheter where the zone has a continuously varying stiffness (or, equivalently a continuously varying critical radius) such that it matches the constraining element at one end and the inherent properties of the catheter at the other. By eliminating any discontinuity along the catheter, the strain relief reduces the potential for kinking. In another variation the continuously varying strain relief can be approximated by a uniform strain relief or a stepped strain relief, each of which reduce the magnitude of the discontinuity between the stiff constraining element and the flexible catheter.

FIG. 4 illustrates a cross-section of a catheter with one embodiment of a strain-relief component 10. Strain relief component 10 is designed to be stiff enough to keep the bending radius in the interface region 200 near the connection to the device wall above the critical radius, yet flexible enough to bend towards the laterally displaced catheter to reduce the bending moment M felt by the portion of the catheter that extends beyond the end of the strain relief.

In the illustrated embodiment, strain relief component 10 is a uniform coating or sleeve that covers catheter's 110 outer surface, changing either or both the effective stiffness of the catheter material or the outer diameter of catheter 110 in region 200. FIG. 4 illustrates catheter 110 after it has been pulled to the side by fill end 110B. Catheter 110 is a flexible, hollow, thin-walled tube. It is surrounded by strain relief 10, which is also a flexible tube-like element. As illustrated, the strain relieved catheter has an increased bend radius in the interface region 200 generally and catheter 110 bends in the direction of the applied force F, as indicated by the arrow, significantly reducing the amount by which the catheter itself must bend in a terminus region 210 where strain relief 10 ends, thus increasing the bending radius, R, above the critical radius R_{C}.

In another embodiment, illustrated in cross-section in FIG. 5, the walls 230 of a tubular strain relief 10A are tapered. This taper creates a continuously varying stiffness and thus bending radius of the catheter in region 200 that corresponds to the variation in wall thickness. Thus, at the constrained end 240 of strain relief 10A the relief 10A is designed to be about as stiff as wall 102 to which it is attached, significantly increasing the bending radius for a given lateral force F near collar 116. Equivalently, in the terminus region 210 of strain relief 10A the added stiffness of the strain relief is almost zero and the strain relief itself has bent to point toward the fill end 110B of the offset catheter, meaning there is no discontinuity in the effective stiffness of tube 110 in the terminus region 210 where the strain relief ends. By providing a continuum of stiffness between the constrained end 240 and the terminus region 210 the strain relief eliminates the catheter bend radius from ever reaching R_{C}.

A tapered-wall embodiment of strain relief 10 can be approximated by a stepped-wall embodiment 10B. For the purposes of comparison, FIGS. 6A and 6B show a right and left side respectively. FIG. 6A illustrates the continuously tapered strain relief 10A of FIG. 5 while the left side figure, FIG. 6B, illustrates a stepped wall embodiment 10B. As its name suggests, a stepped-wall strain relief 10B comprises a wall thickness that varies from thick to thin in stepped manner, wherein the steps 242 can be fabricated in a variety of ways. First, for example, a single, thick strain relief can be cut away to create the desired stepped strain relief. In a second example, the steps can be formed by adding multiple thin layers of wall material, each successively shorter than the preceding layer. In a third fabrication approach the step function can be molded or cast in one piece.

In another embodiment, a tapered strain relief with non-tapered walls may be created by patterning, or spatially modulating, the strain relief's wall. FIG. 7A illustrates a side view of catheter 110 with one variation of a spatially-modulated strain relief 10C. As shown in the illustration, spatially-modulated strain relief 10C comprises a pattern 250 around the exterior of catheter 110. This embodiment, pattern 250, is a zig-zag pattern more clearly seen in FIG. 7B, which shows strain relief 10C unwrapped from catheter 110. That is, the figure illustrates the varying length of the spatially-modulated strain relief wall (as measured from constrained end 240) as a function of angle, θ, around catheter 110.

In the illustrated variation, the spatial modulation is an elongated zig-zag pattern, which can also be described as a series of triangular shapes. Each triangular shape in this example is an isosceles triangle with a narrow base 252 and two elongated sides 254. The width of the base has been selected to be less than one half of the circumference of the catheter and also a fraction of the circumference. That is, there are a whole number, greater or equal to 2, of triangular shapes around the circumference. This pattern is illustrative of desirable properties of a pattern for a spatially-modulated strain relief. First, the wall of the strain relief itself does not have a tapered thickness so it can be fabricated from a simple tube of material. Second, the modulation function comprises only straight lines which are easier to create than curved lines. Third, the modulation pattern repeats multiple times around the circumference of the catheter so there is little or no angular variation in the stiffness of the strain relief around the circumference of the catheter.

In some variations, a spatially-modulated strain relief is a separate component that surrounds the catheter or tube. In another variation, the strain relief is printed directly onto the catheter. The thickness and composition of the ink used in this printing process increases the stiffness of the catheter just as a layer of tubing or molded overcoat would do. For small diameter catheters, cutting or otherwise fabricating the modulated features in a stand-alone, spatially-modulated strain relief is less preferred to simply printing the same features directly on the tubing. Conveniently, adding a printed strain relief can be accomplished with little or no extra expense if the catheter is already being printed with other markings. In some variations these markings are used to estimate the location of the delivery end 110A of the catheter along the gastro-intestinal tract.

### SUMMARY

The present invention relates to fluid delivery systems comprising catheters used to deliver a fluid into a medical device to which the catheter is attached. Also disclosed herein are methods and devices for eliminating the effects angular strain on the catheters that lead to kinking and pinching of the catheter. In particular, a variation of the improved fluid delivery systems described herein include systems for filling medical devices where the device remains in the body after removal of the associated catheter from the device and the body. Variations of the systems and devices also include delivery systems having self-strain-relieving properties; for example, catheters that do not leave behind a potentially problematic strain-relief device on or in the medical device.

The above and other features of the invention including various novel details of construction and combinations of parts, and other advantages, will now be more particularly described with reference to the accompanying drawings and claims. It will be understood that the particular methods and devices conveying the inventive features are shown by way of illustration and not as a limitation of the invention. The principles and features of this invention may be employed in various and numerous embodiments without departing from the scope of the invention.

For example, the present disclosure describes one or more fluid delivery systems for placing a medical device in a patient and delivering a fluid to an enclosed reservoir in the device. In one variation, the system can include a flexible catheter comprising a fill end and a delivery end, wherein said delivery end is configured to be inserted through a wall of the reservoir, the catheter further comprising an initial outer diameter and an initial inner diameter; a region of diametral reduction, where an outer diameter and/or an inner diameter of the region of diametral reduction is reduced compared to the initial outer diameter and/or the initial inner diameter respectively, where the region of diametral reduction is disposed towards the delivery end of the catheter and extending along a length of the catheter, the region starting at or inside the reservoir wall and extending towards the fill end; and wherein the region of diametral reduction comprises a reduction of the initial inner diameter and/or a reduction of the initial outer diameter.

In a variation of the delivery system, the region of diametral reduction has a critical radius, RC and where the region of diametral reduction extends beyond the reservoir wall along a length greater than [πRC]/10 and no less than 2πRC.

In another variation of the delivery system, the region of diametral reduction has a smaller inner and/or outer diameter than the initial inner and/or outer diameter respectively.

Variations of the system include a region of diametral reduction that has an inner and/or outer diameter equal to the initial inner and/or outer diameter respectively of the catheter and a remaining length of the catheter has a greater inner and or outer diameter than the inner and/or outer diameter respectively of the catheter.

The present disclosure includes a flexible catheter having a lumen extending therethrough for delivering the fluid to an enclosed reservoir of a device, the flexible catheter can include a fill end and a delivery end, wherein the delivery end is configured to be coupled to a wall of the enclosed reservoir, the flexible catheter further comprising an initial outer diameter and an initial inner diameter, where the flexible catheter further includes a region of diametral reduction having a passage, where the region of diametral reduction comprises an outer diameter and an inner diameter, where at least one of the outer diameter and the inner diameter is respectively less than the initial outer diameter and the initial inner diameter and where the region of diametral reduction is located adjacent to the delivery end of the flexible catheter and extends along a length of the flexible catheter towards the fill end.

The present disclosure also includes one or more medical devices for positioning in a patient. Such a device can include a balloon member having an internal reservoir, wherein delivery of a fluid into the internal reservoir expands the balloon member; a flexible catheter comprising a fill end and a delivery end, wherein said delivery end is configured to be inserted through a wall of the reservoir, the catheter further comprising an initial outer diameter and an initial inner diameter; a region of diametral reduction, where an outer diameter and/or an inner diameter of the region of diametral reduction is reduced compared to the initial outer diameter, and the initial inner diameter respectively, where the region of diametral reduction is disposed towards the delivery end of the catheter and extending along a length of the catheter, the region starting at or inside the reservoir wall and extending towards the fill end; wherein the region of diametral reduction comprises a reduction of the initial inner diameter and/or a reduction of the initial outer diameter.

In one variation, the present disclosure includes one or more methods of producing a fluid delivery system for filling a medical device placed in a patient using a catheter having a diametrally-reduced region and is configured to deliver a fluid to an enclosed reservoir in the medical device. In one example of a method, the method includes selecting a suitable catheter that meets the engineering requirements for the fluid delivery system; identifying a section of the catheter to produce the diametrally-reduced region; inserting a mandrel into a lumen of the catheter, the mandrel having a diameter equal to a desired inner diameter of the diametrally-reduced region and a length extending past a length of the diametrally-reduced region; applying heat and radially inward-directed pressure to the section; ceasing application of a heat and a radially inward-directed pressure; and removing the mandrel from the catheter.

The method can also comprise protecting the catheter from the heat and pressure outside of the region of diametral reduction.

Another variation of the method further comprises providing a cooling off period prior to removing the mandrel.

A further variation of the method includes applying radially inward-directed pressure by heating a segment of heat-shrink tubing.

The methods can also include protecting the catheter from the heat and pressure outside of the region of diametral reduction by segments of a metal tubing.

Applications for the methods and devices described herein include but are not limited to the devices recited in Table 1. Moreover, the concepts described herein for use with other balloon devices in a wide variety of medical procedures, apart from those shown in Table 1.

TABLE 1
Balloon Device Uses

| Medical Specialty | Procedure |
|---|---|
| Carotid & Neurovascular | Angioplasty, Occlusion |
| ENT | Sinuplasty |
| Cardiovascular | Angioplasty, Stent |
| | Delivery, IVUS, |
| | Vulnerable Plaque |
| | detection |
| Structural Heart | Valvuloplasty, Heart Valve |
| | sizing and dilation, Aortic |
| | pump & Cardioplegia, |
| | Occlusion, Sizing |
| Electrophysiology | Cryoablation |
| EVAR | Sizing, placement, tacking |
| | balloons, endovascular |
| | stent graft delivery |
| GI | Esophageal & biliary |
| | dilation, GI access & stent |
| | placement |
| Venous & AV access | High pressure balloons |
| Iliac | PTA balloons |
| SFA | Long PTA balloons |
| Popliteal, infrapopliteal, | low profile balloons |
| pedal, plantar | |
| MI Orthopaedic | Kyphoplasty |
| Peripheral Vascular | Renal, thrombus aspiration, |
| | stent graft delivery |
| Cosmetic Surgery | Breast Augmentation |

### BRIEF DESCRIPTION OF THE DRAWINGS

The foregoing and other objects, features and advantages of the methods, devices, and systems described are shown the following description in conjunction with the accompanying drawings, in which reference characters refer to the same parts throughout the different views. The drawings are not necessarily to scale; emphasis has instead been placed upon illustrating the principles of the invention.

FIG. 1 illustrates a gastric balloon device.

FIGS. 2A and 2B illustrate a fluid delivery catheter connected to an unfilled balloon.

FIG. 3 illustrates a fluid delivery catheter connected to a filled balloon.

FIG. 4 is a cross-section view of a strain relieved catheter being displaced.

FIG. 5 illustrates a cross-section view one embodiment of a tapered strain relief when the catheter is displaced.

FIG. 6A illustrates a cross-section view of an embodiment of a tapered strain relief.

FIG. 6B illustrates a cross-section view of a stepped embodiment of the tapered strain relief of FIG. 6A.

FIG. 7A is a side view of a spatially modulated strain relief.

FIG. 7B illustrates the spatial modulation pattern of the strain relief of FIG. 7A unwrapped for clarity.

FIG. 8 is a cross-sectional view of an embodiment of a diametrally-reduced catheter.

FIGS. 9A-9E illustrate a process for producing a diametrally-reduced catheter.

FIGS. 10A and 10B illustrate examples of a diametrally-reduced catheter or tubing coupled to a balloon member.

FIG. 11 is a table of experimental results confirming the efficacy of diametral reduction.

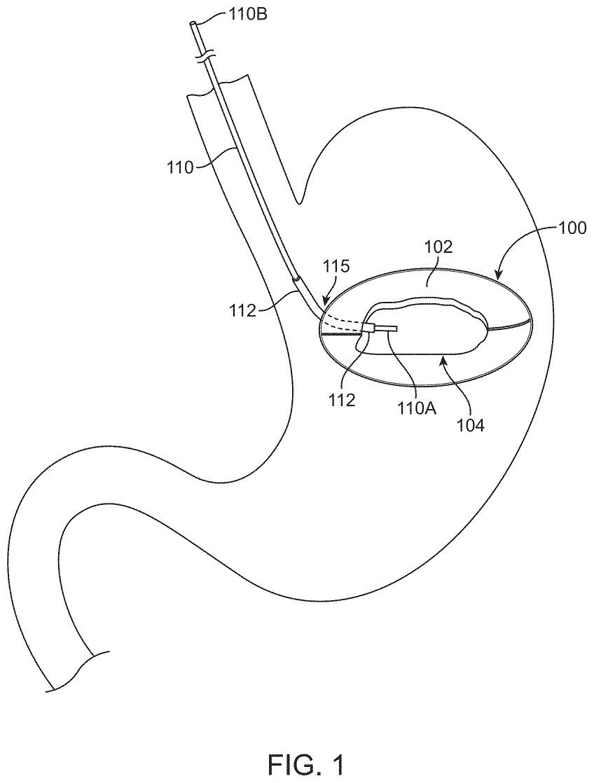

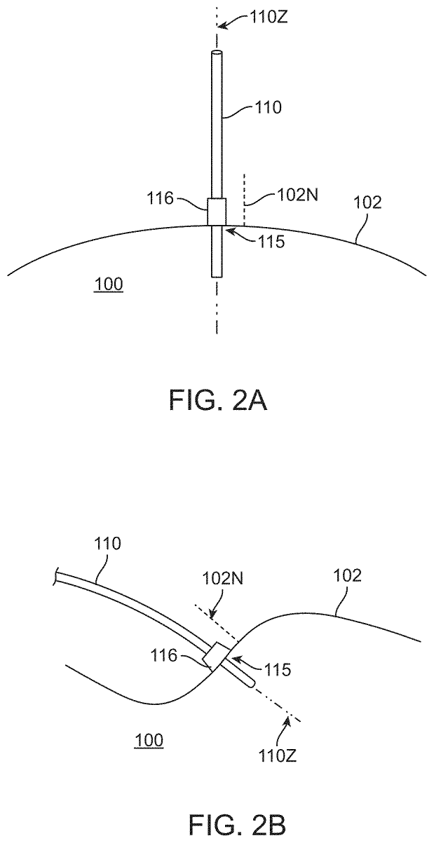

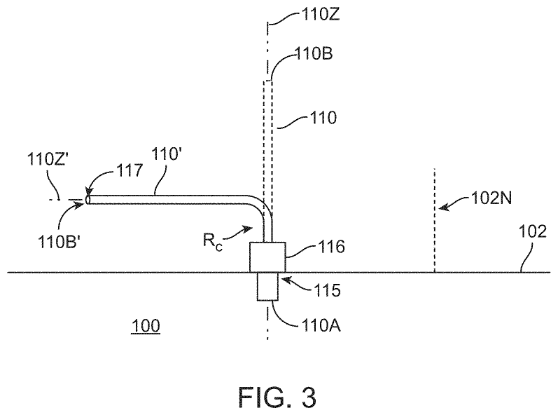

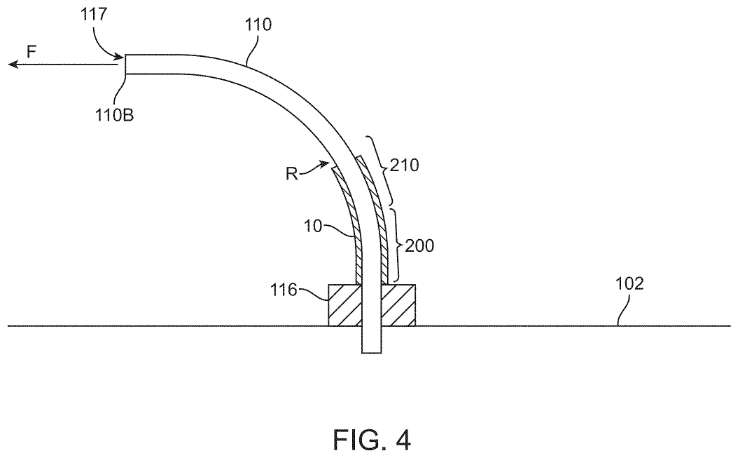

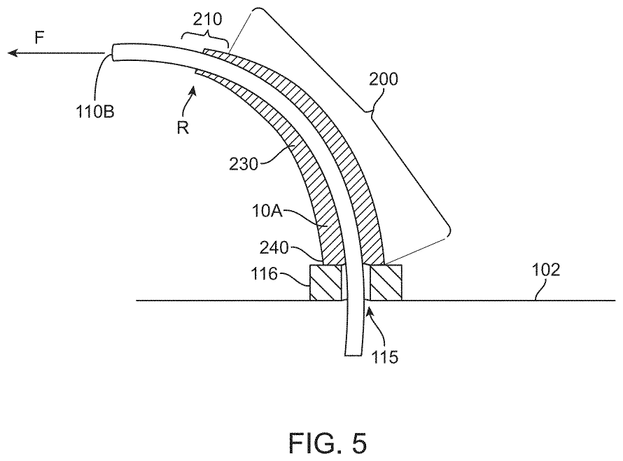

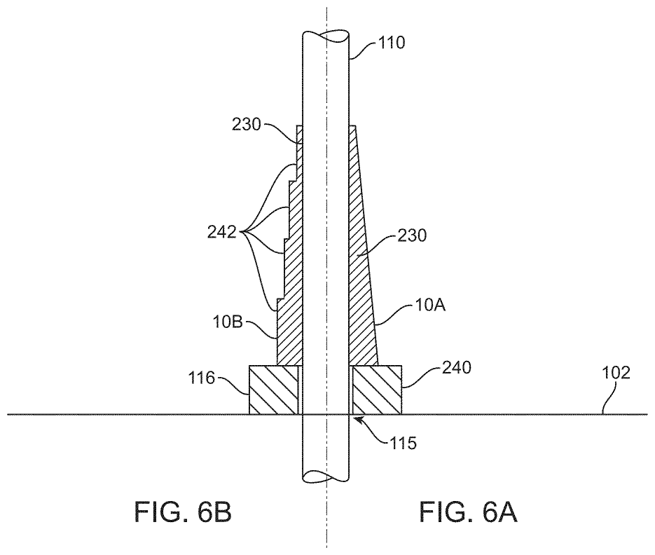

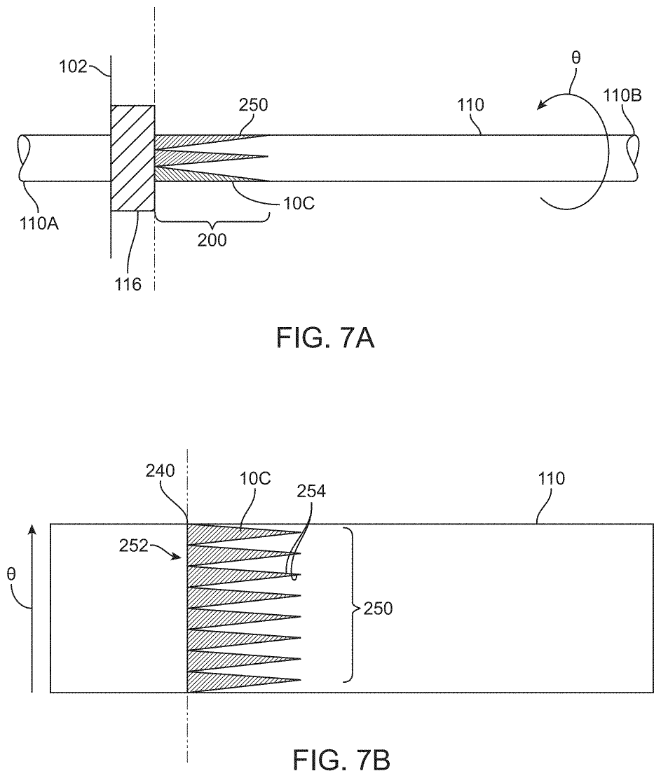

### DETAILED DESCRIPTION OF THE INVENTION

The following illustrations are examples of the invention described herein. It is contemplated that combinations of aspects of specific embodiments or combinations of the specific embodiments themselves are within the scope of this disclosure. The methods, devices, and systems described herein are discussed as being used with a gastric balloon device for convenience for illustrative purposes only. It is intended that the devices, methods, and systems of the present disclosure can be used with other devices where fluid is delivered into/out of the device. For example, such devices can include fluid-inflatable devices that are deployed and inflated with a fluid after insertion into the body. Further, the methods, devices, and system described herein can be used in devices in which a flexible catheter passes through a more rigid barrier.

The angular strain relief approaches described above reducing the likelihood of catheter kinking by mitigating or smoothing the effects of the inherent discontinuity between the stiff constraining element and the flexible catheter, where this mitigation is achieved by adding a mitigating element to the original catheter. Alternatively, it is possible to modify the structure of the catheter itself to reduce its susceptibility to kinking. Two of these catheter modifications are suggested by equation 3, which states that the critical radius depends on three variables—a constant derived from the catheter's material properties (K), the outer diameter of the catheter (D), and the wall thickness of the catheter (D−d) where d is the inner diameter of the catheter. A third modification of the catheter is not related to equation 3. This third structural modification is to change form of the catheter from a cylindrical tube.

The first structural modification to consider is to change a material property of the catheter. For the purposes of this specification, K in equation 3 can be thought of as a measure of springiness. A springy material (e.g., one that is elastically resilient) will allow the outer bend of a catheter to stretch and/or the inner bend to compress.

The second modification suggested by equation 3 is to reduce the catheter's inner and/or outer diameter, creating a “diametrally-reduced” catheter, where an inner diameter and/or an outer diameter of the catheter is reduced relative to another portion of the catheter. This modification is discussed in the next section, Diametral Reduction.

The third structural modification that will reduce a catheter's susceptibility to kinking is to change its geometric structure from a pure cylindrical tube to a tube in which the walls are not uniform. The critical radius for this geometric structure is not described by equation 3, which only applies to a cylindrical tube.

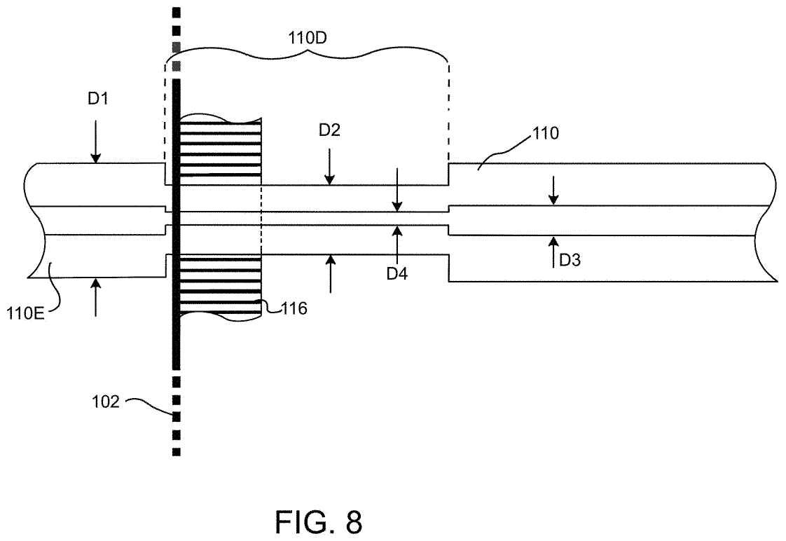

Diametral reduction, specifically reduction of the inner diameter (ID), is a method of decreasing the critical radius of a catheter. Additionally, as shown in equation 3, reducing the OD can also decrease the critical radius as long as the wall thickness, equal to ½ (OD−ID) is not reduced so much as to counteract the effect of the diametral reductions. That is, since the critical radius is inversely proportional to the wall thickness it grows rapidly as the wall thickness approaches zero. One variation of a diametrally-reduced catheter is illustrated in cross-section in FIG. 8. In this variation a catheter 110 has been treated to reduce the diameter (both ID and OD) over a section of its length. Reduced diameter section 110D is the section of the catheter intended to pass through, or be held by, the relatively stiffer constraining element 116. Section 110D can be located anywhere along catheter 110 and in some variations of the catheter it may be beneficial to dispose section 110D displaced away from an end of the catheter to leave an unmodified, and therefor stiffer, leading catheter section 110E. This leading section 110E, because it is generally stiffer than reduced diameter section 110D, may be easier than section 110D to thread through the lumen in constraining element 116.

As shown in FIG. 8, catheter 110 has an initial OD of D1 and initial ID of D3. In reduced diameter section 110D the OD is reduced to D2 while the ID is reduced to D4. Typically, the target value of D4 is determined by the minimum system flow rate requirement while the minimum achievable critical radius is achieved with an experimentally or computer-aided design (CAD) determined target D2_{T}.

The efficacy of diametral reduction to decrease the critical radius was demonstrated using a thermal diametral reduction process. In one example, a 0.070-inch OD, 0.054-inch ID catheter was modified to have a target ID of 0.046-inch over an approximately 1-inch section of the catheter. In one variation, the portion of the catheter which passes through the constraining element 116 is located about 1-inch from the end of the catheter. To create a sufficient length to bend as needed, a 1-inch reduced diameter section was created.

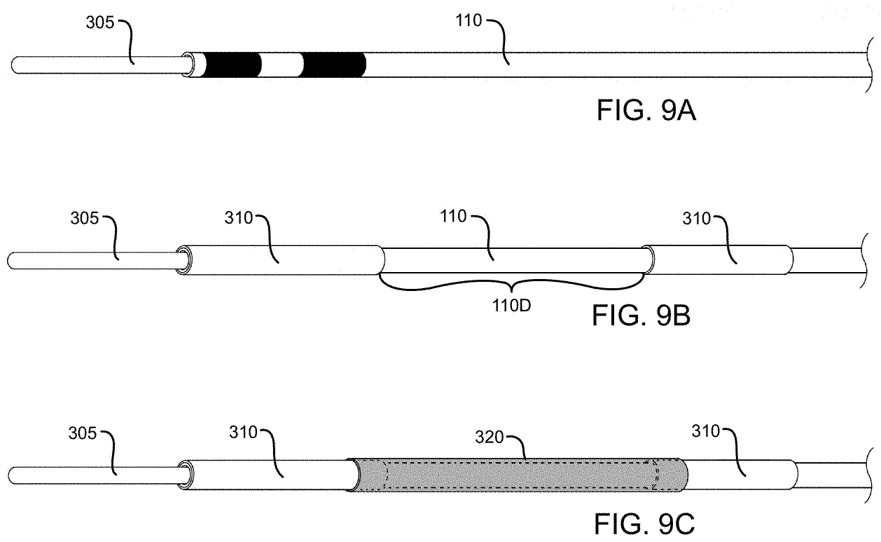

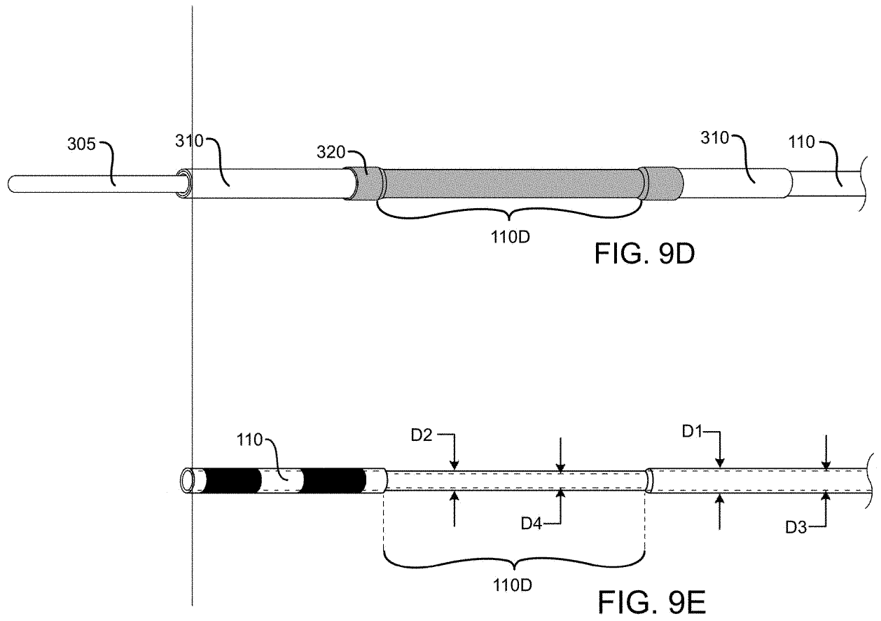

In this variation, an initial step in the diametral reduction process is illustrated in FIG. 9A wherein a Teflon®-coated 304 stainless steel (304SS) 0.045-inch diameter mandrel 305 has been inserted into the 0.054-inch lumen of catheter 110. The diameter of mandrel 305 defines the reduced internal diameter D4 of the diametrally-reduced catheter. Mandrel 305 has a length sufficient to extend into the lumen of catheter 110 approximately 1-inch beyond the planned diametrally-reduced section 110D.

Another step of the process is shown in FIG. 9B and comprises sliding two segments of 304SS tubing 310 over the exterior of catheter 110, where tubing 310 has an 0.072-inch ID to conveniently fit over the conduit's 0.070-inch initial OD [D1] and a 0.095-inch OD. Tubing segments 310 are thermo-mechanical shields 310 to protect the catheter from diametral reduction outside of section 110D. As shown, one shield is located with its leading edge coincident with the end of catheter 110 while the second shield is located with its leading edge disposed to leave a gap equal to section 110D. In this example the gap is approximately 1-inch.

FIG. 9C illustrates a third step of the process in which a polyolefin heat shrink tube 320 with 0.10″ ID is slid onto the catheter, over both shields 310, covering section 110D and overlapping both shields 310 slightly. After tube 320 is in place, heat is applied to the polyolefin tube 320 until it shrinks, compressing catheter 110, which is also heated, so that the catheter 110 shrinks inwardly against Teflon-coated mandrel 305, as shown in FIG. 9D. During this example process, the OD of section 110D was reduced to 0.064-inches. It may be noted that shields 310 prevent tube 320 from compressing conduit 110 outside of the intended diametrally-reduced section 110D.

After allowing time to cool, the polyolefin heat shrink tubing is carefully cut/torn away and shields 310 are removed, as shown in FIG. 9E. As shown, the catheter 110 includes an initial outer diameter D1 and initial inner diameter D3 (in a lumen of the catheter), where the diametrally-reduced section 110D includes an outer diameter D2 and a passage having an inner diameter D4. For this example, the catheter was kept on the mandrel for at least 10 minutes to avoid unwanted additional shrinkage.

In one experiment, eight sample diametrally-reduced catheters were produced. These catheters and eight control catheters cut from the same stock were installed in a fixture to measure their kink resistance when bent in a small radius. That is, the tests performed provided an estimate the critical radius reduction of the diametrally-reduced catheters relative to the control catheters. More specifically, the test procedure and fixture measured the bend diameter at which flow through the catheter was reduced by a specified percentage, that is, it measured a functional kink diameter. This is a functional measure of kink resistance since flow through the catheter is the primary specification for the catheter.

The test method performed to assess kink resistance of the heat-shrunk catheters compared to unmodified catheters comprised bending the test object into a decreasing radius arc while water was pumped through the catheter at a constant pressure. Kink resistance was quantified by measuring the arc radius at which flow is reduced by 50% compared to the same catheter segment when not bent. For the usual uses of a catheter, the “50% flow rate radius” measured in this test is more useful than an actual measurement of the critical radius. The 50% flow reduction, while arbitrary, is a valid indication of kink-resistance. The heat-shrunk catheters were made using the process detailed above.

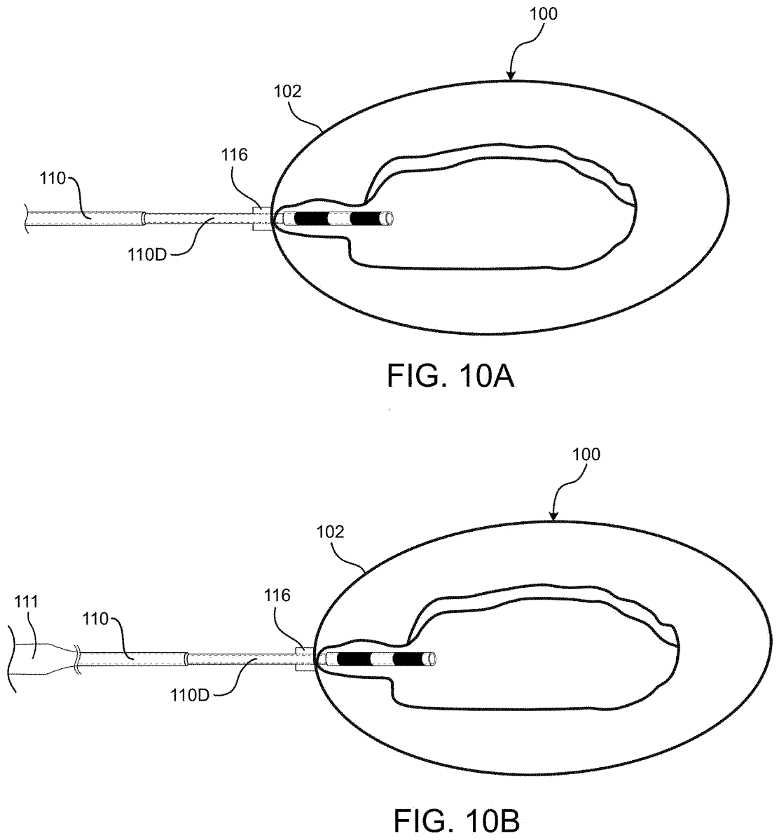

FIGS. 10A and 10B illustrate examples of a catheter or tubing 110 having a diametrally-reduced region 110D adjacent to a wall 102 of a balloon-device 110. Variations of the configuration can include the diametrally-reduced region 110D extending into the balloon 100, stopping at the wall 102, or stopping in a constraining element 116. The diametrally-reduced region 110D shown is for purposes of illustration and can have a shorter or longer length than shown. FIG. 10B illustrates a catheter 110 having a region 111 of increased internal and/or external diameter 111 at a location spaced from the diametrally—reduced region 110.

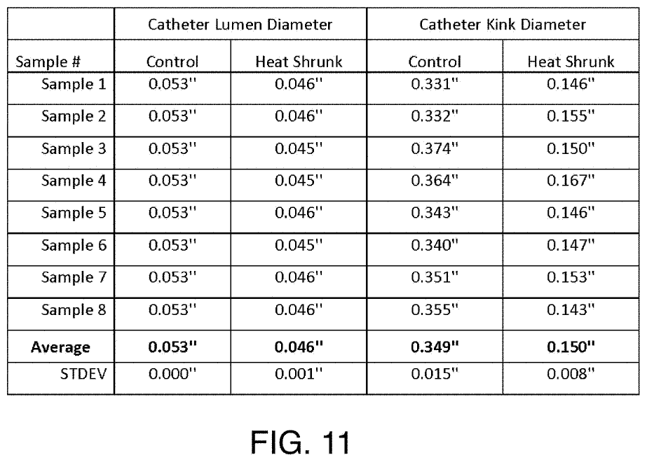

FIG. 11 is a table of results from this assessment. As shown in the table, the thermal diametral reduction technique produced a very consistence reduced inner diameter of 0.046 inches, reduced from the original (and control) ID of 0.053 inches. The outer diameter was also reduced from the original (and control) OD of 0.070 inches to a reduced OD of 0.064 inches. More important than the specific ID and OD reductions is the reduction in the kink diameter. From this assessment we see that these diametrally-reduced catheters have a kink diameter approximately 42% of the control catheters. During testing, it was found that the absolute flow rate of the unkinked reduced diameter catheter was only 4%-5% lower than the unkinked control catheters, indicating that the functional impact of reducing the ID of these catheters was minimal. It is believed the apparent disagreement between the predictions of equation 3 and the kink diameter determined by flow rate is due to the difference between the definition of R_{c}, which is the radius at which kinking starts, and the kink diameter, which is based on 50% flow reduction.

It should be noted that the diametrally-reduced catheter described herein can be used in with a region of strain relief, where the region of diametral reduction 110D may coincide with interface region 200 (that is, region 200 may cover all or most of region 110D) or be located to start at or to extend beyond terminus region 210 in the direction of catheter fill end 110B.

We claim:

1. A fluid delivery system for delivering a fluid to an enclosed reservoir in a device, the fluid delivery system comprising:
a flexible catheter having a lumen extending therethrough for delivering the fluid to the enclosed reservoir, the flexible catheter comprising a fill end and a delivery end, wherein the delivery end is configured to be coupled to a wall of the enclosed reservoir, the flexible catheter further comprising an initial outer diameter and an initial inner diameter, where the flexible catheter further includes a region of diametral reduction having a passage;
where the region of diametral reduction comprises an outer diameter and an inner diameter, where at least one of the outer diameter and the inner diameter is respectively less than the initial outer diameter and the initial inner diameter, where the region of diametral reduction is located adjacent to the delivery end of the flexible catheter and extends along a length of the flexible catheter towards the fill end.

2. The fluid delivery system of claim 1, where a first end of the region of diametral reduction is located within the enclosed reservoir and a second end of the region of diametral reduction is located exterior to the enclosed reservoir.

3. The fluid delivery system of claim 1, where a first end of the region of diametral reduction is located at a wall surrounding the enclosed reservoir and a second end of the region of diametral reduction is located exterior to the enclosed reservoir.

4. The fluid delivery system of claim 1, wherein the region of diametral reduction has a critical radius and where the region of diametral reduction extends beyond the wall of the enclosed reservoir along a length at least greater than (pi times the critical radius) divided by 10 and at least no less than two times pi times the critical radius.

5. A medical device for positioning in a patient:
a balloon member having an internal reservoir, wherein delivery of a fluid into the internal reservoir expands the balloon member;
a flexible catheter having a lumen extending therethrough, the flexible catheter comprising a fill end and a delivery end, wherein the delivery end is coupled to the balloon member such that the lumen is in fluid communication with the internal reservoir, the flexible catheter further comprising an initial outer diameter and an initial inner diameter comprising the lumen having, where the flexible catheter further includes a region of diametral reduction having a passage and located adjacent to the balloon member; and
the region of diametral reduction including an outer diameter and an inner diameter comprising the passage, where at least one of the outer diameter and the inner diameter is respectively less than the initial outer diameter and the initial inner diameter.

6. A method of producing a fluid delivery system for a medical device using a catheter configured to deliver a fluid to an enclosed reservoir in the medical device in a patient, the method comprising:
selecting a suitable catheter that meets a requirement for the fluid delivery system;
identifying a section of the catheter to produce the diametrally-reduced region;
inserting a mandrel into a lumen of the catheter, the mandrel having a diameter equal to a desired diameter of the diametrally-reduced region and a length extending past a length of the diametrally-reduced region;
applying a heat and a radially inward-directed pressure to the section;
ceasing application of the heat and the radially inward-directed pressure; and
removing the mandrel from the catheter.

7. The method of claim 6, where applying the heat and the radially inward-directed pressure occurs simultaneously.

8. The method of claim 6, further comprising protecting the catheter from the heat and pressure at one or more lengths of the catheter adjacent to the section.

9. The method of claim 6, further comprising removing the mandrel after a cooling-off period.

10. The method of claim 6, wherein applying the heat and the radially inward-directed pressure includes use of a heat-shrink tubing.

11. The method of claim 6 wherein the catheter is protected from the heat and the radially inward-directed pressure outside of the region of diametral reduction by segments of a metal tubing.

12. A fluid delivery system for delivering a fluid to an enclosed reservoir in a device, the fluid delivery system comprising:
a flexible catheter having a lumen extending therethrough, the flexible catheter comprising a fill end and a delivery end, wherein the delivery end is configured to be inserted through a wall of the enclosed reservoir, the flexible catheter further comprising an initial outer diameter and an initial inner diameter comprising the lumen, where the flexible catheter further includes a region of diametral reduction;
where the region of diametral reduction comprises an outer diameter being less than the initial outer diameter and an inner diameter being less than the initial inner diameter, where the region of diametral reduction is located adjacent to the delivery end of the flexible catheter and extends along a length of the flexible catheter towards the fill end, the region starting at or inside the wall of the enclosed reservoir and extending towards the fill end.
